(19) United States
(12) Patent Application Publication
(10) Pub. No.: US 2011/0152807 A1
(43) Pub. Date: Jun. 23, 2011

(54) ARTICLE INCLUDING A BIODEGRADABLE TEXTILE MATERIAL

(75) Inventors: Chien-Ming Huang, Nantun District (TW); Yu-Kai Huang, Taiping City (TW)

(73) Assignee: MINIMA TECHNOLOGY CO., LTD., Taichung City (TW)

(21) Appl. No.: 12/822,602

(22) Filed: Jun. 24, 2010

(30) Foreign Application Priority Data
Dec. 22, 2009 (TW) 098144183

(51) Int. Cl. A61F 13/15; A47L 25/00

(52) U.S. Cl. 604/365; 604/367; 15/227

(57) ABSTRACT

An article includes: a biodegradable textile material which includes a liquid absorbent layer made of a fiber material selected from rayon, cotton, linen, and synthetic fibers; and a liquid-impermeable layer bonded to the liquid absorbent layer, and made from a biodegradable composition including polybutylene succinate as a major component.

### CROSS-REFERENCE TO RELATED APPLICATION

This application claims priority of Taiwanese application no. 098144183, filed on Dec. 22, 2009.

### BACKGROUND OF THE INVENTION

1. Field of the Invention

This invention relates to an article, more particularly to an article including a biodegradable textile material.

2. Description of the Related Art

At present, commercial textile sanitary products, such as diapers or sanitary napkins, are made of a textile material having both liquid-absorbing and liquid-impermeable properties. The textile material generally includes a non-woven fabric base layer made of polyester, and a water-impermeable layer attached to the fabric base layer and made of polyethylene.

Although this kind of textile material may exhibit the water-absorbing and water-impermeable properties by virtue of the fabric base layer and the water-impermeable layer, polyester and polyethylene respectively used for fabrication of these two layers are not biodegradable polymers. In addition, this kind of textile material is usually used for manufacturing disposable products. If consumption of these disposable products is not discontinued, the accumulated non-biodegradable wastes would be tremendous and thus, require a relatively high cost of treatment and tend to cause environmental problems.

### SUMMARY OF THE INVENTION

Therefore, an object of the present invention is to provide an article that can overcome the aforesaid drawback associated with the prior art.

According to one aspect of the present invention, an article comprises: a biodegradable textile material which includes a liquid absorbent layer; and a liquid-impermeable layer bonded to the liquid absorbent layer, and made from a biodegradable composition including polybutylene succinate as a major component.

According to another aspect of this invention, a glove-shaped rag comprises a glove body made of a biodegradable textile material which includes a liquid absorbent layer, and a liquid-impermeable layer bonded to the liquid absorbent layer and made from a biodegradable composition including polybutylene succinate as a major component.

### BRIEF DESCRIPTION OF THE DRAWINGS

Other features and advantages of the present invention will become apparent in the following detailed description of the preferred embodiment of this invention, with reference to the accompanying drawings, in which:

FIG. 1 is a fragmentary sectional view of the preferred embodiment of an article including a biodegradable textile material according to this invention; and

FIG. 2 is a perspective view of the preferred embodiment of a glove-shaped wiping rag including a biodegradable textile material according to this invention.

### DETAILED DESCRIPTION OF THE PREFERRED EMBODIMENT

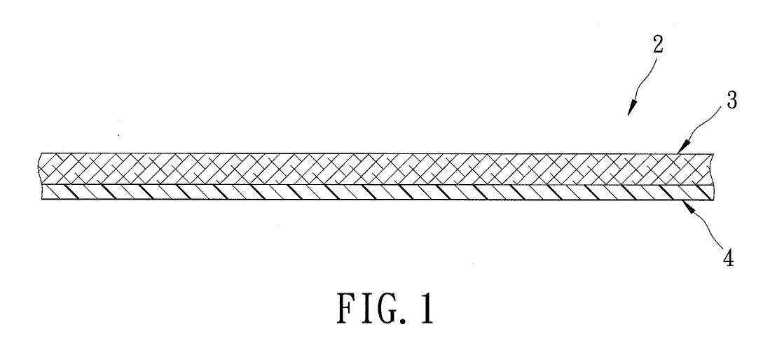

Referring to FIG. 1, an article of the preferred embodiment according to this invention includes a biodegradable textile material 2.

The biodegradable textile material 2 includes a liquid absorbent layer 3 and a liquid-impermeable layer 4.

Preferably, the liquid absorbent layer 3 is made of a fiber material selected from the group consisting of rayon, cotton, linen, and combinations thereof. Due to the fiber material, the liquid absorbent layer 3 can provide liquid absorbing property, and comfortable and soft feel upon touch.

The liquid-impermeable layer 4 is bonded to the liquid absorbent layer 3, and is made from a biodegradable composition including polybutylene succinate (PBS) as a major component. Preferably, the liquid-impermeable layer 4 is bonded to the liquid absorbent layer 3 by coating, laminating and the like. In one preferred embodiment, the liquid-impermeable layer 4 has a layer thickness ranging from 12 μm to 35 μm.

It is worth mentioning that the liquid-impermeable layer 4 bonded to the liquid absorbent layer 3 by coating, laminating and the like may be made from a biodegradable composition include solely polybutylene succinate. Alternatively, the biodegradable composition may include the major component of polybutylene succinate (PBS) and further include a minor component selected from the group consisting of polylactic acid (PLA), polybutylene adipate-co-terephthalate (PBAT), poly(caprolactone) (PCL), and combinations thereof. The weight ratio of the major component to the minor component may be varied depending on practical application. Preferably, based on a total weight of the biodegradable composition, the major component is in an amount ranging from 70 wt % to 100 wt %, and the minor component is in an amount ranging from 0 wt % to 30 wt %.

In one preferred embodiment, by virtue of addition of a small amount of polybutylene adipate-co-terephthalate, flowability or the melting index of the biodegradable composition can be modified so as to increase the viscoelasticity thereof and so as to improve adhesion of the liquid-impermeable layer 4 to the liquid absorbent layer 3. In another preferred embodiment, addition of polylactic acid may increase rigidity of the liquid-impermeable layer 4 so as to improve the dimensional stability of the article. In yet another preferred embodiment, by virtue of addition of poly(caprolactone), flexibility and processability of the liquid-impermeable layer 4 can be improved. In addition, since the major and minor components both are biodegradable polymers, they tend to be easily mixed with each other due to relatively good compatibility. Compared to the conventional non-biodegradable polymer, the major and minor components have a lower melting point and can be melted into an easily formable state at a relatively low temperature, thereby alleviating damage to the liquid absorbent layer 3 when the liquid-impermeable layer 4 is bonded thereto.

Besides, mechanical properties of polybutylene succinate (PBS) are similar to those of polypropylene and acrylonitrile-butadiene-styrene copolymer (ABS) and superior to other biodegradable materials. The PBS has a heat deflection temperature (HDT) as high as about 90° C. and retains the waterproofing effect of polymers. Therefore, the liquid-impermeable layer 4 made of polybutylene succinate can prevent liquid from permeating into and damaging the liquid absorbent layer 3 so as to impart the liquid-impermeable effect to the article. In addition, since the article including the biodegradable textile material 2 is biodegradable, the environmental protection requirements can be met and the cost of waste treatment can be reduced.

Polybutylene succinate used in the liquid-impermeable layer 4 may be selected from the commercial products, such as the products obtained from Mitsubishi Chemical Corporation under trade names: GS pla, AZ91, AZ71, and AZ61 and their combinations.

Preferably, the article made of the biodegradable textile material 2 is a rag, a diaper, a sanitary napkin, or a pet pad. When the article is one of the diaper, the sanitary napkin, and the pet pad, the liquid absorbent layer 3 is used to serve as a surface layer to face the user, and the liquid-impermeable layer 4 is provided to abase layer for preventing the absorbed liquid from leaking out.

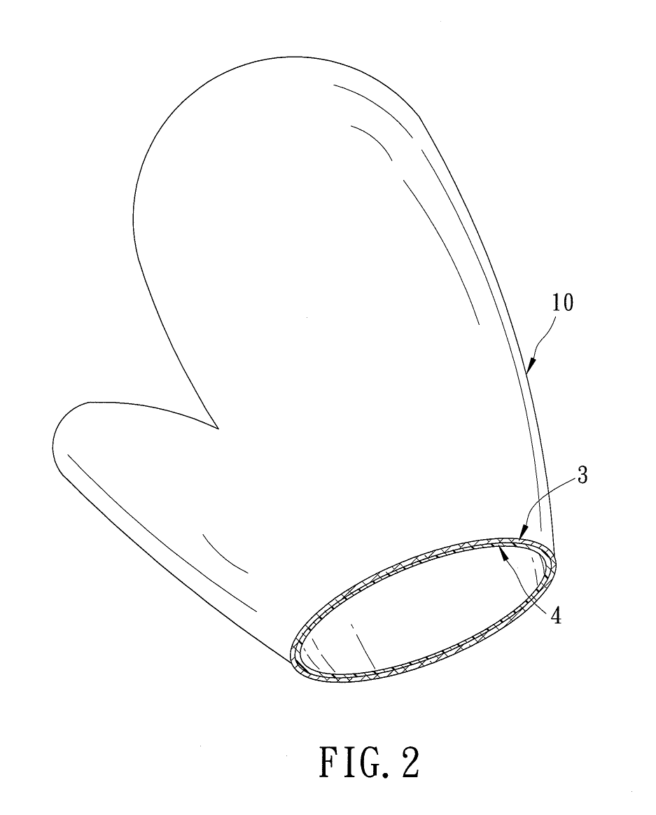

Referring to FIG. 2, the article including the biodegradable textile material of this invention is a glove-shaped wiping rag 10 that includes a glove body having an interior layer and an exterior layer, and made of the biodegradable textile material 2.

The liquid absorbent layer 3 is used as the exterior layer for cleaning and wiping out dirt and contaminants, and the liquid-impermeable layer 4 is used as the interior layer for protecting a user's hand from being contaminated.

According to this invention, by virtue of fabricating the article, especially the disposable article, with the biodegradable textile material 2, the article is biodegradable such that, the environmental protection requirements can be met and the cost of waste treatment can be reduced.

With the invention thus explained, it is apparent that various modifications and variations can be made without departing from the spirit of the present invention. It is therefore intended that the invention be limited only as recited in the appended claims.

What is claimed is:

1. An article comprising:
a biodegradable textile material which includes
a liquid absorbent layer; and
a liquid-impermeable layer bonded to said liquid absorbent layer, and made from a biodegradable composition including polybutylene succinate as a major component.

2. The article of claim 1, wherein said liquid absorbent layer is made of a fiber material selected from the group consisting of rayon, cotton, linen, and combinations thereof.

3. The article of claim 1, wherein said biodegradable composition further includes a minor component, based on a total weight of the biodegradable composition, the major component is in an amount ranging from 70 wt % to 100 wt %, and the minor component is in an amount ranging from 0 wt % to 30 wt %.

4. The article of claim 3, wherein said minor component is selected from the group consisting of polylactic acid, polybutylene adipate/terephthalate, poly(caprolactone) and combinations thereof.

5. The article of claim 1, wherein said liquid-impermeable layer has a layer thickness ranging from 12 μm to 35 μm.

6. The article of claim 1, wherein said liquid-impermeable layer is coated on said liquid absorbent layer.

7. The article of claim 1, wherein said liquid-impermeable layer is laminated to said liquid absorbent layer.

8. The article of claim 1, wherein the article is a rag.

9. The article of claim 1, wherein the article is a diaper.

10. The article of claim 1, wherein the article is a sanitary napkin.

11. The article of claim 1, wherein the article is a pet pad.

12. A glove-shaped wiping rag comprising:
a glove body having an interior layer and an exterior layer, and made of a biodegradable textile material which includes
a liquid absorbent layer; and
a liquid-impermeable layer bonded to said liquid absorbent layer, and made from a biodegradable composition including polybutylene succinate as a major component;
wherein said liquid absorbent layer is used as said exterior layer, and said liquid-impermeable layer is used as said interior layer.

13. The glove-shaped wiping rag of claim 12, wherein said liquid absorbent layer is formed of a fiber material selected from the group consisting of rayon, cotton, linen, and combinations thereof.

14. The glove-shaped wiping rag of claim 12, wherein said biodegradable composition further includes a minor component, based on a total weight of the biodegradable composition, the major component is in an amount ranging from 70 wt % to 100 wt %, and the minor component is in an amount ranging from 0 wt % to 30 wt %.

15. The glove-shaped wiping rag of claim 14, wherein said minor component is selected from the group consisting of polylactic acid, polybutylene adipate/terephthalate, poly(caprolactone) and combinations thereof.

16. The glove-shaped wiping rag of claim 12, wherein said liquid-impermeable layer has a layer thickness ranging from 12 μm to 35 μm.
